(19) United States
(12) Patent Application Publication
(10) Pub. No.: US 2024/0242806 A1
(43) Pub. Date: Jul. 18, 2024

(54) SYSTEM FOR MANAGING PHARMACOTHERAPY AND METHOD OF OPERATING THE SAME

(71) Applicant: ODN Co., Ltd., Seoul (KR)

(72) Inventor: Sang Youl RHEE, Seoul (KR)

(73) Assignee: ODN Co., Ltd., Seoul (KR)

(21) Appl. No.: 18/392,692

(22) Filed: Dec. 21, 2023

(30) Foreign Application Priority Data
Dec. 22, 2022 (KR) 10-2022-0181571

(51) Int. Cl. G16H 20/10; G16H 10/60; G16H 50/30; G16H 70/40

(52) U.S. Cl. CPC G16H 20/10; G16H 10/60; G16H 50/30; G16H 70/40

(57) ABSTRACT

The present disclosure relates to a system for managing pharmacotherapy and a method of operating the same. The system for managing pharmacotherapy according to one embodiment includes an information receiver for receiving profile information and clinical examination information about a patient; an obesity index calculator for calculating the obesity index of the patient based on the profile information; a status classifier for classifying the obesity status of the patient into a preset obesity stage based on the calculated obesity index and the clinical examination information; a treatment information generator for generating pharmacotherapy information corresponding to the classified obesity stage and providing the generated pharmacotherapy information to the terminal of a medical professional; and an efficacy verifier for verifying the efficacy of the prescribed drug based on the measurement values of weight information and vital factors of the patient at a first time point at which the medical professional prescribed a drug to the patient and a second time point at which a preset treatment period has elapsed from the first time point.

### CROSS-REFERENCE TO RELATED APPLICATION

This application claims priority to Korean Patent Application No. 10-2022-0181571, filed on Dec. 22, 2022, in the Korean Intellectual Property Office, the disclosure of which is incorporated herein by reference.

### BACKGROUND OF THE DISCLOSURE

### Field of the Disclosure

The present disclosure relates to a system for managing pharmacotherapy and a method of operating the same, and more particularly, to a technical idea for managing pharmacotherapy in obese patients.

### Description of the Related Art

According to the World Health Organization (WHO), the number of obese people around the world has more than doubled over the past few decades. In addition, it is predicted that by 2030, more than half of adults around the world will be overweight or obese.

Treatment options such as diet, exercise, behavior modification, pharmacotherapy, and bariatric surgery are being used to effectively manage obesity. In particular, obesity management through pharmacotherapy is recommended for obese people who do not achieve adequate weight loss through behavior modification.

Such pharmacotherapy is performed by medical professionals, and for more efficient pharmacotherapy, the need for development of a system that supports a medical professional's decision-making process of drug prescription and dosage is increasing.

### RELATED ART DOCUMENTS

### Patent Documents

Korean Patent Application Publication No. 10-2020-0136950, “SYSTEM FOR MANAGING PERSONALIZED PHARMACOTHERAPY AND METHOD OF MANAGING PERSONALIZED PHARMACOTHERAPY”
Korean Patent No. 10-2397683, “METHOD AND SYSTEM FOR MICROBIAL PHARMACOGENOMICS”

### SUMMARY OF THE DISCLOSURE

Therefore, the present disclosure has been made in view of the above problems, and it is an object of the present disclosure to provide a system for managing pharmacotherapy that supports the prescription of drugs that match the patient's obesity status by providing pharmacotherapy information based on the classification results of the obesity index and obesity status of a patient and a method of operating the system.

It is another object of the present disclosure to provide a system for managing pharmacotherapy that evaluates the efficacy and effects of a drug prescribed to a patient and supports optimizing the drug and dosage prescribed to the patient and a method of operating the system.

In accordance with one aspect of the present disclosure, provided is a system for managing pharmacotherapy including an information receiver for receiving profile information and clinical examination information about a patient; an obesity index calculator for calculating an obesity index of the patient based on the profile information; a status classifier for classifying obesity status of the patient into a preset obesity stage based on the calculated obesity index and the clinical examination information; a treatment information generator for generating pharmacotherapy information corresponding to the classified obesity stage and providing the generated pharmacotherapy information to a terminal of a medical professional; and an efficacy verifier for verifying efficacy of the prescribed drug based on measurement values of weight information and vital factors of the patient at a first time point at which the medical professional prescribed a drug to the patient and a second time point at which a preset treatment period has elapsed from the first time point.

According to one aspect, the profile information may include at least one information of age, gender, height, weight, disease history, disease-related family history, vital signs, physical activity amount, and dietary pattern of the patient.

According to one aspect, the obesity index calculator may calculate a body mass index (BMI) of the patient as the obesity index based on the profile information.

According to one aspect, the clinical examination information may include results of clinical examination for at least one disease of angina, myocardial infarction, heart failure, thrombosis, coronary artery disease, coronary artery obstructive pulmonary disease, dyspnea, and stroke of the patient.

According to one aspect, the clinical examination information may include evaluation information about at least one of metabolic risk, physical disability, and psychological problems according to results of clinical examination of the patient.

According to one aspect, the status classifier may classify obesity status of the patient as stage 0 when the evaluation information is marked as ‘none (Nil)’, may classify obesity status of the patient as stage 1 when the evaluation information is marked as ‘mild’, and may classify obesity status of the patient as stage 2 when the evaluation information is marked as ‘moderate’.

According to one aspect, the status classifier may classify obesity status of the patient as stage 3 when the evaluation information is marked as ‘considerable’, and may classify obesity status of the patient as stage 4 when the evaluation information is marked as ‘serious’.

According to one aspect, the treatment information generator may generate the pharmacotherapy information including at least one information of long-term medication information and short-term medication information corresponding to the classified obesity stage.

According to one aspect, the efficacy verifier may provide notification information for any one of continuation, discontinuation, and change of the prescribed drug based on verification results of efficacy of the prescribed drug to a terminal of the medical professional.

According to one aspect, the efficacy verifier may generate the notification information for continuing the prescribed drug and dosage of the prescribed drug when a difference between weight information at the second time point and weight information at the first time point exceeds a preset first threshold, and may generate the notification information for changing the prescribed drug to another drug and adjusting dosage of the prescribed drug when a difference between the weight information does not exceed the first threshold.

According to one aspect, the efficacy verifier may generate the notification information for continuing or discontinuing taking the prescribed drug based on a difference between a measurement value of a vital factor at the second time point and a measurement value of a vital factor at the first time point, wherein the vital factor includes at least one of a first vital factor, a second vital factor, and a third vital factor.

According to one aspect, the efficacy verifier may generate the notification information for continuing the prescribed drug and a dosage of the prescribed drug when a difference between a measurement value of a first vital factor at the second time point and a measurement value of a first vital factor at the first time point does not exceed a preset second threshold.

According to one aspect, when a difference between a measurement value of a first vital factor at the second time point and a measurement value of a first vital factor at the first time point exceeds a preset second threshold, and a difference between a measurement value of a second vital factor at the second time point and a measurement value of a second vital factor at the first time point does not exceed the second threshold, the efficacy verifier may generate the notification information for continuing or changing a dosage of the prescribed drug.

According to one aspect, when a difference between a measurement value of a first vital factor at the second time point and a measurement value of a first vital factor at the first time point exceeds a preset second threshold, a difference between a measurement value of a second vital factor at the second time point and a measurement value of a second vital factor at the first time point exceeds the second threshold, and a difference between a measurement value of a third vital factor at the second time point and a measurement value of a third vital factor at the first time point does not exceed the second threshold, the efficacy verifier may generate the notification information for continuing or changing a dosage of the prescribed drug.

According to one aspect, when a difference between a measurement value of a first vital factor at the second time point and a measurement value of a first vital factor at the first time point exceeds a preset second threshold, a difference between a measurement value of a second vital factor at the second time point and a measurement value of a second vital factor at the first time point exceeds the second threshold, and a difference between a measurement value of a third vital factor at the second time point and a measurement value of a third vital factor at the first time point exceeds the second threshold, the efficacy verifier may generate the notification information for discontinuing taking the prescribed drug.

According to one aspect, the first vital factor may include at least one of white blood cells (WBCs), hemoglobins, platelets, creatinine, epidermal growth factor receptor (eGFR), aspartate aminotransferase (AST), and alanine aminotransferase (ALT).

According to one aspect, the second vital factor may include at least one of red blood cells (RBCs), γ-glutamyl transferase (GGT), blood urea nitrogen (BUN), cholesterol, high-density lipoprotein (HDL), low-density lipoprotein (LDL), glycated hemoglobin (HbA1c), and bioimpedance.

According to one aspect, the third vital factor may include at least one of hematocrit, neutrophils, lymphocytes, monocytes, eosinophils, basophils, uric acid, proteins, albumin, and triglyceride.

According to one aspect, the treatment information generator may generate the pharmacotherapy information including initial drug recommendation information for the patient by using a reinforcement learning-based drug recommendation model that uses at least one of the classified obesity stage, the profile information, and the clinical examination information as input.

In accordance with another aspect of the present disclosure, provided is a method of operating a system for managing pharmacotherapy, the method including receiving profile information and clinical examination information about a patient by an information receiver; calculating an obesity index of the patient based on the profile information by an obesity index calculator; classifying obesity status of the patient into a preset obesity stage based on the calculated obesity index and the clinical examination information by a status classifier; generating pharmacotherapy information corresponding to the classified obesity stage and providing the generated pharmacotherapy information to a terminal of a medical professional by a treatment information generator; and verifying, by an efficacy verifier, efficacy of the prescribed drug based on measurement values of weight information and vital factors of the patient at a first time point at which the medical professional prescribed a drug to the patient and a second time point at which a preset treatment period has elapsed from the first time point.

### BRIEF DESCRIPTION OF THE DRAWINGS

The above and other objects, features and other advantages of the present disclosure will be more clearly understood from the following detailed description taken in conjunction with the accompanying drawings, in which:

FIG. 1 is a diagram for more specifically explaining a system for managing pharmacotherapy according to one embodiment;

FIG. 2 is a diagram for more specifically explaining a system for managing pharmacotherapy according to one embodiment;

FIGS. 3A to 3C are diagrams for explaining examples of providing information and notification information about a patient in a system for managing pharmacotherapy according to one embodiment; and

FIG. 4 is a flowchart for explaining a method of operating a system for managing pharmacotherapy according to one embodiment.

### DETAILED DESCRIPTION OF THE DISCLOSURE

Specific structural and functional descriptions of embodiments according to the concept of the present disclosure disclosed herein are merely illustrative for the purpose of explaining the embodiments according to the concept of the present disclosure. Furthermore, the embodiments according to the concept of the present disclosure can be implemented in various forms and the present disclosure is not limited to the embodiments described herein.

The embodiments according to the concept of the present disclosure may be implemented in various forms as various modifications may be made. The embodiments will be described in detail herein with reference to the drawings. However, it should be understood that the present disclosure is not limited to the embodiments according to the concept of the present disclosure, but includes changes, equivalents, or alternatives falling within the spirit and scope of the present disclosure.

The terms such as “first” and “second” are used herein merely to describe a variety of constituent elements, but the constituent elements are not limited by the terms. The terms are used only for the purpose of distinguishing one constituent element from another constituent element. For example, a first element may be termed a second element and a second element may be termed a first element without departing from the teachings of the present disclosure.

It should be understood that when an element is referred to as being “connected to” or “coupled to” another element, the element may be directly connected or coupled to the other element or intervening elements may be present. In contrast, when an element is referred to as being “directly connected to” or “directly coupled to” another element, there are no intervening elements present. Other words used to describe the relationship between elements or layers should be interpreted in a like fashion (e.g., “between,” versus “directly between,” “adjacent,” versus “directly adjacent,” etc.).

The terms used in the present specification are used to explain a specific exemplary embodiment and not to limit the present inventive concept. Thus, the expression of singularity in the present specification includes the expression of plurality unless clearly specified otherwise in context. Also, terms such as “include” or “comprise” should be construed as denoting that a certain characteristic, number, step, operation, constituent element, component or a combination thereof exists and not as excluding the existence of or a possibility of an addition of one or more other characteristics, numbers, steps, operations, constituent elements, components or combinations thereof.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, preferred embodiments of the present disclosure will be described in detail with reference to the accompanying drawings. However, the scope of the present disclosure is not limited by these embodiments. Like reference numerals in the drawings denote like elements.

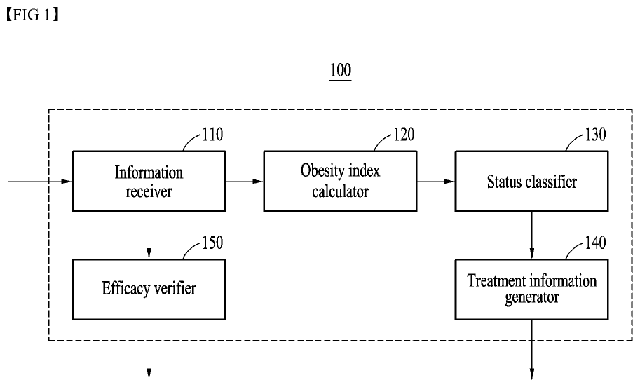

FIG. 1 is a diagram for more specifically explaining a system for managing pharmacotherapy according to one embodiment.

Referring to FIG. 1, a system 100 for managing pharmacotherapy according to one embodiment may support the prescription of a drug that matches the obesity status of a patient by providing pharmacotherapy information based on the classification results of the obesity index and obesity status of the patient.

In addition, the system 100 for managing pharmacotherapy may evaluate the efficacy and effects of a drug prescribed to a patient and support optimizing the drug and dosage prescribed to the patient.

The system 100 for managing pharmacotherapy may include an information receiver 110, an obesity index calculator 120, a status classifier 130, a treatment information generator 140, and an efficacy verifier 150.

The information receiver 110 according to one embodiment may receive profile information and clinical examination information about a patient. Here, the profile information and the clinical examination information may be profile information and clinical examination information at a first time point, and the clinical examination information at the first time point may include the measurement value of a vital factor for a patient at the first time point.

For example, the profile information may include at least one information of age, gender, height, weight, disease history, disease-related family history, vital signs, physical activity amount, and dietary pattern of a patient.

In addition, the clinical examination information may include results of clinical examination for at least one disease of angina, myocardial infarction, heart failure, thrombosis, coronary artery disease, coronary artery obstructive pulmonary disease, dyspnea, and stroke of a patient. However, the clinical examination information according to one embodiment not limited to the above-mentioned diseases and may also include results from various clinical pathology tests.

Specifically, the clinical examination information may include results of at least one test of a physiological function test (e.g., electroencephalography and electrocardiogram), a biochemical test (e.g., liver function test, kidney test, blood sugar test, and electrolyte test), a hematology test (e.g., anemia test, blood type test, and blood cell test), an immuno-serology test (e.g., hepatitis test), and a urine chemistry test (e.g., kidney function test and pregnancy test) performed for a patient.

In addition, the clinical examination information may include evaluation information about at least one of metabolic risk, physical disability, and psychological problems according to results of clinical examination of a patient.

The obesity index calculator 120 according to one embodiment may calculate the obesity index of a patient based on profile information.

For example, the obesity index calculator 120 may calculate the body mass index (BMI) of a patient as an obesity index based on height information and weight information among profile information.

Specifically, the BMI index stands for body mass index, is a formula that estimates the amount of fat using height and weight, and is an indicator that reflects body fat percentage and health risk. The obesity class of Koreans and the obesity class of foreigners according to BMI index may be determined based on Tables 1 and 2 below, respectively.

TABLE 1

| Obesity class | Body mass index (kg/m²) |
|---|---|
| Underweight | <18.5 |
| Normal | 18.5~22.9 |
| Pre-obesity stage | 23~24.9 |
| Stage 1 obesity (First class) | 25~29.9 |
| Stage 2 obesity (Second class) | 30~34.9 |
| Stage 3 obesity (Third class) | ≥35 |

TABLE 2

| Obesity class | Body mass index (kg/m²) |
|---|---|
| Underweight | <18.5 |
| Normal | 18.5~24.9 |
| Pre-obesity stage | 25.0~29.9 |
| Stage 1 obesity (First class) | 30~34.9 |
| Stage 2 obesity (Second class) | 35.0~39.9 |
| Stage 3 obesity (Third class) | ≥40 |

The status classifier 130 according to one embodiment may classify the obesity status of a patient as a preset obesity stage based on a calculated obesity index and clinical examination information.

For example, when the calculated obesity index is higher than the value corresponding to stage 1 obesity (i.e., based on Koreans, when the BMI index is 25 or higher), the status classifier 130 may classify the obesity status of a patient as a preset obesity stage. Here, the preset obesity stage may be a stage based on the Edmonton Obesity Staging System (EOSS).

According to one aspect, when obvious obesity-related risk factors, physical symptoms, psychopathology, functional limitations, and/or impaired well-being are absent, the status classifier 130 may classify the obesity status as stage 0. When obesity-related subclinical risk factors, mild physical symptoms, mild psychopathology, mild functional limitations, and/or impaired well-being are present, the status classifier 130 may classify the obesity status as stage 1.

In addition, when established obesity-related chronic diseases, moderate limitations in activities of daily living, and/or impaired well-being are present, the status classifier 130 may classify the obesity status as stage 2. When damage to end organs, serious psychopathology, serious functional limitations, and/or impaired well-being are present, the status classifier 130 may classify the obesity status as stage 3. When severe disability due to obesity-related chronic diseases (potentially terminal), psychopathology disorder, functional limitations, and/or impaired well-being are present, the status classifier 130 may classify the obesity status as stage 4.

Specifically, when evaluation information (i.e., evaluation information about at least one of metabolic risk, physical disability, and psychological problems) included in clinical examination information is marked as ‘none (Nil)’, the status classifier 130 may classify the obesity status of a patient as stage 0. When evaluation information is marked as ‘mild’, the status classifier 130 may classify the obesity status of a patient as stage 1. When evaluation information is marked as ‘moderate’, the status classifier 130 may classify the obesity status of a patient as stage 2.

In addition, when evaluation information is marked as ‘considerable’, the status classifier 130 may classify the obesity status of a patient as stage 3. When evaluation information is marked as ‘serious’, the status classifier 130 may classify the obesity status of a patient as stage 4.

According to one aspect, when evaluation information according to metabolic risk, evaluation information according to physical disability, and evaluation information according to psychological problems are different from each other, the status classifier 130 may classify the patient's obesity status in the following order of priority: stage 4, stage 3, stage 2, stage 1, and stage 0.

The treatment information generator 140 according to one embodiment may generate pharmacotherapy information corresponding to the classified obesity stage, and may provide the generated pharmacotherapy information to the terminal of a medical professional.

For example, the treatment information generator 140 may generate pharmacotherapy information including information about a preset recommended drug list corresponding to a classified obesity stage, that is, stages 0 to 4, and a recommended dosage for each recommended drug.

That is, the treatment information generator 140 may support the prescription of a drug that matches the patient's obesity status by providing pharmacotherapy information including information about a recommended drug list and a recommended dosage for each recommended drug to the terminal of a medical professional.

According to one aspect, the treatment information generator 140 may generate pharmacotherapy information including at least one information of long-term medication information and short-term medication information corresponding to a classified obesity stage.

In addition, the treatment information generator 140 may generate pharmacotherapy information including initial drug recommendation information about a patient using a reinforcement learning-based drug recommendation model that uses at least one of a classified obesity stage, profile information, and clinical examination information as input.

Specifically, the treatment information generator 140 may perform databaseization of obesity stage classification information, profile information, clinical examination information, drug prescription information, and efficacy verification results for prescribed drugs for a plurality of patients, may build a drug recommendation model through reinforcement learning on the database information, and may provide initial drug recommendation information that matches the user's obesity status using the built drug recommendation model.

For example, the drug recommendation model according to one embodiment may provide initial drug recommendation information including information about the drugs listed in Tables 3 to 5 described later. However, the drug recommendation model is not limited thereto, and may provide information on previously known drugs related to obesity management in addition to the drugs listed in Tables 3 to 5.

The efficacy verifier 150 according to one embodiment may verify the efficacy of a prescribed drug based on the measurement values of weight information and vital factors of a patient at a first time point at which a medical professional prescribed a drug to the patient and a second time point at which a preset treatment period has elapsed from the first time point.

The information receiver 110 may receive profile information at the second time point including weight information at the second time point and clinical examination information at the second time point including the measurement value of a vital factor at the second time point.

Here, the vital factor is an indicator for evaluating the body's response to a drug administered to a patient. Depending on the risk of an obese patient, the vital factors may include a first vital factor to be considered first, a second vital factor to be considered next, and a third vital factor to be considered last.

For example, the first vital factor may include at least one of white blood cells (WBCs), hemoglobins, platelets, creatinine, epidermal growth factor receptor (eGFR), aspartate aminotransferase (AST), and alanine aminotransferase (ALT).

In addition, the second vital factor may include at least one of red blood cells (RBCs), γ-glutamyl transferase (GGT), blood urea nitrogen (BUN), cholesterol, high-density lipoprotein (HDL), low-density lipoprotein (LDL), glycated hemoglobin (HbA1c), and bioimpedance.

In addition, the third vital factor may include at least one of hematocrit, neutrophils, lymphocytes, monocytes, eosinophils, basophils, uric acid, proteins, albumin, and triglyceride.

In addition, the measurement value of the vital factor may include at least one of a numerical value and a count value of the vital factor derived through clinical examination.

According to one aspect, based on the verification results of efficacy of a drug prescribed by a medical professional to a patient, the efficacy verifier 150 may provide notification information for any one of continuation, discontinuation, and change of the prescribed drug to a terminal of the medical professional.

That is, the efficacy verifier 150 may support optimization of a drug and a drug dosage prescribed to a patient by evaluating the efficacy and effects of the drug prescribed to the patient and providing notification information for any one of continuation, discontinuation, and change of the prescribed drug to a medical professional.

According to one aspect, when a difference between weight information at a second time point and weight information at a first time point exceeds a preset first threshold, the efficacy verifier 150 may generate notification information for continuing a prescribed drug and the dosage of the prescribed drug. When a difference between the weight information does not exceed the first threshold, the efficacy verifier 150 may generate notification information for changing a prescribed drug to another drug and adjusting the dosage of the prescribed drug.

Preferably, the efficacy verifier 150 may generate notification information by determining whether the change rate of weight information in a preset treatment period (interval from the first time point to the second time point) exceeds a preset first threshold ratio (e.g., 5%).

According to one aspect, the efficacy verifier 150 may generate notification information for continuing or discontinuing taking a prescribed drug based on a difference between the measurement value of a vital factor at a second time point and the measurement value of a vital factor at a first time point.

Specifically, when a difference between the measurement value of a first vital factor at a second time point and the measurement value of a first vital factor at a first time point does not exceed a preset second threshold, the efficacy verifier 150 may generate notification information for continuing a prescribed drug and the dosage of the prescribed drug.

In addition, when a difference between the measurement value of a first vital factor at a second time point and the measurement value of a first vital factor at a first time point exceeds a second threshold, and a difference between the measurement value of a second vital factor at a second time point and the measurement value of a second vital factor at a first time point does not exceed the second threshold, the efficacy verifier 150 may generate notification information for continuing or changing the dosage of a prescribed drug.

In addition, when a difference between the measurement value of a first vital factor at a second time point and the measurement value of a first vital factor at a first time point exceeds a preset second threshold, a difference between the measurement value of a second vital factor at a second time point and the measurement value of a second vital factor at a first time point exceeds the second threshold, and a difference between the measurement value of a third vital factor at a second time point and the measurement value of a third vital factor at a first time point does not exceed the second threshold, the efficacy verifier 150 may generate notification information for continuing or changing the dosage of a prescribed drug.

In addition, when a difference between the measurement value of a first vital factor at a second time point and the measurement value of a first vital factor at a first time point exceeds a preset second threshold, a difference between the measurement value of a second vital factor at a second time point and the measurement value of a second vital factor at a first time point exceeds the second threshold, and a difference between the measurement value of a third vital factor at a second time point and the measurement value of a third vital factor at a first time point exceeds the second threshold, the efficacy verifier 150 may generate notification information for discontinuing taking a prescribed drug.

Preferably, the efficacy verifier 150 may generate notification information by determining whether the change rate of the measurement value of a vital factor (first to third vital factors) in a preset treatment period (interval from the first time point to the second time point) exceeds a preset second threshold range (e.g., 30%).

In addition, the status classifier 130 may classify the patient's condition at a second time point as a preset obesity stage, and the efficacy verifier 150 may generate notification information for continuing or discontinuing taking a prescribed drug based on the classified obesity stage at the second time point.

For example, when the obesity stage at the second time point is from stage 0 to stage 2, the efficacy verifier 150 may generate notification information for continuing a prescribed drug and the dosage of the prescribed drug.

In addition, when the obesity stage at the second time point is stage 3 or stage 4, the efficacy verifier 150 may generate notification information for discontinuing taking a prescribed drug.

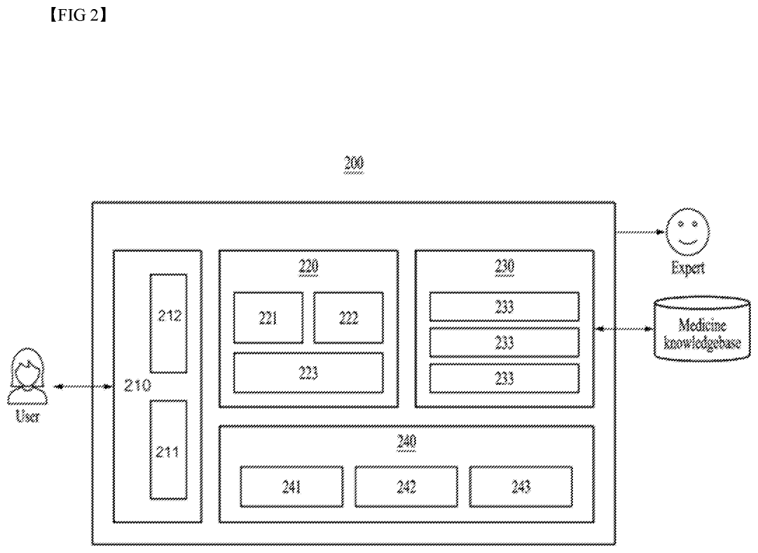

The system 100 for managing pharmacotherapy according to one embodiment will be described more specifically with reference to FIG. 2.

FIG. 2 is a diagram for more specifically explaining a system for managing pharmacotherapy according to one embodiment.

Referring to FIG. 2, a system 200 for managing pharmacotherapy according to one embodiment may provide personalized pharmacotherapy through personal profile, health level assessment, prescription recommendations, and follow-up management.

The system 200 for managing pharmacotherapy may include an information manager 210, a health level estimator 220, a medication prescription manager 230, and a follow-up manager 240.

The information manager 210 according to one embodiment may receive profile information and clinical examination information about a patient. That is, the information manager 210 may be the information receiver 110 of FIG. 1.

According to one aspect, the information manager 210 may receive and manage profile information about a patient through an information management function. The information manager 210 may further include an information builder 211 and an information logging device 212.

Specifically, the information management function is a function that manages demographic changes, visit information, and recommended test types for users suspected of being obese or obese users (i.e., patients), and may provide the ability to manage logs to track changes in the patient's weight and BMI.

In addition, the information builder 211 may provide the ability to collect and register initial information related to age, gender, height, weight, disease, family history, and vital signs through a dedicated app or web. The information logging device 212 may provide the function of recording and managing all information obtained when a patient visits a dedicated app or web or visits a medical institution after a registration process in the information builder 211 is completed. Specifically, the information logging device 212 may provide the ability to compare demographic changes and clinical trial results based on logs generated each time a patient visits.

The health level estimator 220 according to one embodiment may identify the patient's obesity grade, BMI level, and status of recommended essential factors based on clinical trial results, and may determine whether the patient has a comorbidity based on the identification results. The health level estimator 220 may include an obesity index identifier 221, a comorbidity identifier 222, and a vital factor evaluator 223.

The obesity index identifier 221 may identify the patient's obesity index based on the patient's height information, weight information, and gender information. That is, the obesity index identifier 221 may be the obesity index calculator 120 of FIG. 1.

For example, the obesity index identifier 221 may calculate the patient's BMI index, which is derived through calculations based on the patient's height information, weight information, and gender information, as an obesity index.

The comorbidity identifier 222 may provide the function of evaluating metabolic, physical, and psychological parameters to determine optimal obesity treatment, and through this, the patient's obesity status may be classified into a preset obesity stage. That is, the comorbidity identifier 222 may be the status classifier 130 of FIG. 1.

Specifically, when obvious obesity-related risk factors, physical symptoms, psychopathology, functional limitations, and/or impaired well-being are absent, the comorbidity identifier 222 may classify the obesity status as stage 0. When obesity-related subclinical risk factors, mild physical symptoms, mild psychopathology, mild functional limitations, and/or impaired well-being are present, the comorbidity identifier 222 may classify the obesity status as stage 1.

In addition, when established obesity-related chronic diseases, moderate limitations in activities of daily living, and/or impaired well-being are present, the comorbidity identifier 222 may classify the obesity status as stage 2. When damage to end organs, serious psychopathology, serious functional limitations, and/or impaired well-being are present, the comorbidity identifier 222 may classify the obesity status as stage 3. When severe disability due to obesity-related chronic diseases (potentially terminal), psychopathology disorder, functional limitations, and/or impaired well-being are present, the comorbidity identifier 222 may classify the obesity status as stage 4.

The vital factor evaluator 223 may perform a function to obtain clinical insight into physical indicators to evaluate the patient's physical response to drug intake. The vital factor evaluator 223 may classify a plurality of vital factors into a first vital factor, a second vital factor, and a third vital factor according to importance and manage the measurement value of each vital factor. That is, the vital factor evaluator 223 may be included in the efficacy verifier 150 of FIG. 1.

For example, the first vital factor may include at least one of white blood cells (WBCs), hemoglobins, platelets, creatinine, epidermal growth factor receptor (eGFR), aspartate aminotransferase (AST), and alanine aminotransferase (ALT).

In addition, the second vital factor may include at least one of red blood cells (RBCs), γ-glutamyl transferase (GGT), blood urea nitrogen (BUN), cholesterol, high-density lipoprotein (HDL), low-density lipoprotein (LDL), glycated hemoglobin (HbA1c), and bioimpedance.

In addition, the third vital factor may include at least one of hematocrit, neutrophils, lymphocytes, monocytes, eosinophils, basophils, uric acid, proteins, albumin, and triglyceride.

The medication prescription manager 230 according to one embodiment may generate pharmacotherapy information, and may provide the generated pharmacotherapy information to the terminal of a medical professional in charge of patients. That is, the medication prescription manager 230 may be the treatment information generator 140 of FIG. 1.

In addition, the medication prescription manager 230 may be linked to a medical knowledge-based database (medicine knowledgebase) that stores big data on obesity-related drugs to generate pharmacotherapy information.

Specifically, the medication prescription manager 230 may support more accurate treatment and prescriptions by a medical professional by generating pharmacotherapy information including information according to a prescribed drug and the minimum and maximum dosage of the drug in response to the patient's condition analysis results. The medication prescription manager 230 may include a comorbidity analyzer 231, a drug recommender 232, and a prescription recommender 233.

The comorbidity analyzer 231 may determine a plurality of drugs corresponding to the obesity status of a patient corresponding to a classified obesity stage, that is, any one of stages 0 to 4.

The drug recommender 232 may generate pharmacotherapy information including at least one information of long-term medication information and short-term medication information as shown in Table 3 below based on a plurality of drugs corresponding to the obesity status of a patient.

However, the drugs according to one embodiment are not limited to the drugs listed in Table 3 below, and new incretin-based drugs that are currently on the market or will be released in the future may also be used.

TABLE 3

| Long Term Medication | Short Term Medication |
|---|---|
| Orlistate | Phentermine |
| Natrexonce | Diethylpropion |
| ER-bupropion | Phendimetrazine |
| Liraglutide | Mazindol |
| Phentermine-topirate ER | |
| Semaglutide (oral) | |
| Semaglutide (injection) | |
| Tirzepatide (injection) | |

The prescription recommender 233 may generate pharmacotherapy information including information on drug prescription contraindicators as shown in Table 4 below and information on the dosages of drugs as shown in Table 5 below.

TABLE 4

| Medicine | Contraindicators |
|---|---|
| Phentermine | 1. Hyperactive thyroid patient |
| | 2. Diabetic patient |
| | 3. Patient with psychotic disorder |
| | 4. Patient with alcoholism |
| | 5. Drug abuse patient |
| | 6. Patient with glaucoma or increased eye pressure |
| | 7. Patient with significant uncontrolled hypertension |
| | 8. Patient with mild hypertension |
| Diethylpropion | 1. Hyperactive thyroid patient |
| | 2. Patient with type 2 diabetes |
| | 3. Patient with type 1 diabetes |
| | 4. Patient with toxic psychosis |
| | 5. Patient with alcoholism |
| | 6. Drug abuse patient |
| | 7. Patient with glaucoma or increased eye pressure |
| | 8. Patient with hypertension |
| Phendimetrazine | 1. Patient with a history of heart disease |
| | 2. Patient with coronary artery disease |
| | 3. Patient with cardiac arrhythmia |
| | 4. Patient with congestive heart failure |
| | 5. Patient with uncontrolled hypertension |
| Mazindol | 1. Patient with glaucoma and arrhythmia |
| | 2. Patient in a nervous state |
| | 3. Patient with a history of drug abuse |
| | 4. Patient taking monoamine oxidase inhibitor |
| Orlistat | 1. Patient with hypersensitivity to orlistat or ingredients thereof |
| | 2. Patient with chronic malabsorption |
| | 3. Patient with cholestasis |
| | 4. Patient with anorexia and bulimia |
| | 5. Pregnant patient |
| | 6. Patient with severe renal impairment |
| Naltrexone ER- | 1. Patient with uncontrolled hypertension |
| bupropion | 2. Patient with seizure symptoms |
| | 3. Patient with eating disorder |
| | 4. Patient with opioid addiction |
| | 5. Pregnant patient |
| | 6. Patients taking opioid or other forms of bupropion |
| Liraglutide | 1. Patient with a personal or family history of certain types of thyroid |
| | cancer, especially thyroid C-cell tumors such as medullary thyroid |
| | carcinoma (MTC) or multiple endocrine tumor syndrome type 2 |
| | (MEN 2) |
| Phentermine- | 1. Patient with acute myopia |
| topiramate ER | 2. Patient with secondary angle-closure glaucoma |
| | 3. Patient with hyperthyroidism |
| | 4. Patient taking monoamine oxidase (MAO) inhibitor |

TABLE 5

| Medine | Dosage |
|---|---|
| Phentermine | 8 mg/day to 30 mg/day |
| Diethylpropion | 25 mg/day to 75 mg/day |
| Phendimetrazine | 35 mg/day to 105 mg/day |
| Mazindol | 1 mg/day to 3 mg/day |
| Orlistat | 120 mg/day to 360 mg/day |
| Naltrexone ER-bupropion | 32 mg/day to 360 mg/day |
| ER-bupropion | 32 mg/day to 360 mg/day |
| Liraglutide | 0.6 mg/day to 3.0 mg/day |
| Phentermine-topiramate ER | 3.75 mg/23 mg to 15 mg/92 mg |
| Semaglutide (oral) | 3 mg/day to 14 mg/day |
| Semaglutide (injection) | 0.25 mg/week to 1 mg/week |
| Tirzepatide (injection) | 2.5 mg/week to 15 mg/week |

The follow-up manager 240 according to one embodiment may verify the efficacy of a prescribed drug based on the measurement values of weight information and vital factors of a patient at a first time point at which a medical professional prescribed the drug to the patient and a second time point at which a preset treatment period has elapsed from the first time point. That is, the follow-up manager 240 may be the efficacy verifier 150 of FIG. 1.

That is, after a patient takes a drug prescribed by a medical professional, the follow-up manager 240 may evaluate the impact and effectiveness of the drug. The follow-up manager 240 may include a treatment period analyzer 241, an efficacy evaluator 242, and a safety gauge judgment device 243.

Specifically, the treatment period must take at least 12 weeks until an individual feels any significant abnormal symptoms or adverse effects in terms of psychological or physical condition. The treatment period analyzer 241 may generate notification information for consideration of further processing (treatment) when the number of weeks (NO.Of.Weeks) is more than 12 weeks by mapping with weeks, considering the difference between the current date (CurrentDate) (i.e., second time point) and the start date (StartDate) (i.e., first time point) of a prescribed drug, as shown in Equation 1 below.

$$\frac{CurrentDate - StartDate}{7} = No . Of . Weeks \qquad [ Equation 1 ]$$

When a difference between weight information at a second time point and weight information at a first time point exceeds a preset first threshold, the efficacy evaluator 242 may generate notification information for continuing a prescribed drug and the dosage of the prescribed drug. When a difference between the weight information does not exceed the first threshold, the efficacy evaluator 242 may generate notification information for changing a prescribed drug to another drug and adjusting the dosage of the prescribed drug.

That is, when the patient's weight change exceeds the first threshold during a treatment period, the efficacy evaluator 242 may provide information for continuing a prescribed drug. When the patient's weight change is less than the first threshold during a treatment period, the efficacy evaluator 242 may provide information for changing a drug and the dosage of the drug.

Preferably, the efficacy evaluator 242 may determine whether the weight change rate (% age Change Of Weight) of a patient calculated through calculation of weight information (OriginalWeight) at a first time point and weight information (CurrentWeight) at a second time point, as shown in Equation 2 below, exceeds a first threshold ratio (e.g., 5%), and may generate notification information.

$$\frac{OriginalWeight - CurrentWeight}{OriginalWeight} = \% age Change Of Weight \qquad [ Equation 2 ]$$

Based on a difference between the measurement value of a vital factor at a second time point and the measurement value of a vital factor at a first time point, the safety gauge judgment device 243 may generate notification information for continuing or discontinuing taking a prescribed drug.

That is, the safety gauge judgment device 243 may monitor the physical symptoms of a patient taking a prescribed drug through clinical examination, and may provide support to change or discontinue the prescribed drug according to the monitoring results.

Specifically, when a difference between the measurement value of a first vital factor at a second time point and the measurement value of a first vital factor at a first time point does not exceed a preset second threshold, the safety gauge judgment device 243 may generate notification information for continuing a prescribed drug and the dosage of the prescribed drug.

In addition, when a difference between the measurement value of a first vital factor at a second time point and the measurement value of a first vital factor at a first time point exceeds a second threshold, and a difference between the measurement value of a second vital factor at a second time point and the measurement value of a second vital factor at a first time point does not exceed the second threshold, the safety gauge judgment device 243 may generate notification information for continuing or changing the dosage of a prescribed drug.

In addition, when a difference between the measurement value of a first vital factor at a second time point and the measurement value of a first vital factor at a first time point exceeds a preset second threshold, a difference between the measurement value of a second vital factor at a second time point and the measurement value of a second vital factor at a first time point exceeds the second threshold, and a difference between the measurement value of a third vital factor at a second time point and the measurement value of a third vital factor at a first time point does not exceed the second threshold, the safety gauge judgment device 243 may generate notification information for continuing or changing the dosage of a prescribed drug.

In addition, when a difference between the measurement value of a first vital factor at a second time point and the measurement value of a first vital factor at a first time point exceeds a preset second threshold, a difference between the measurement value of a second vital factor at a second time point and the measurement value of a second vital factor at a first time point exceeds the second threshold, and a difference between the measurement value of a third vital factor at a second time point and the measurement value of a third vital factor at a first time point exceeds the second threshold, the safety gauge judgment device 243 may generate notification information for discontinuing taking a prescribed drug.

Preferably, the safety gauge judgment device 243 may determine whether the measurement value change rate (% age Change Critical Factor) of the vital factor of a patient calculated through calculation of the measurement value (Original Critical Factor Value) of a vital factor at a first time point and the measurement value (Current Critical Factor Value) of a vital factor at a second time point, as shown in Equation 3 below, exceeds a second threshold ratio (e.g., 30%), and may generate notification information.

$$\frac{\begin{matrix}OriginalCriticalFactorValue - \\ CurrentCriticalFactorValue\end{matrix}}{OriginalCriticalFactorValue} = \% age ChangeCriticalFactor \qquad [ Equation 3 ]$$

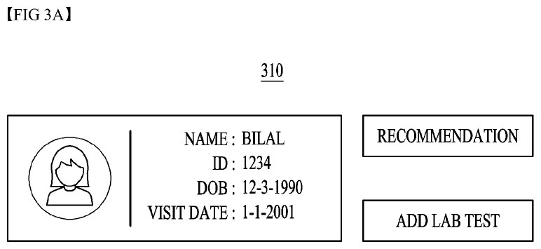

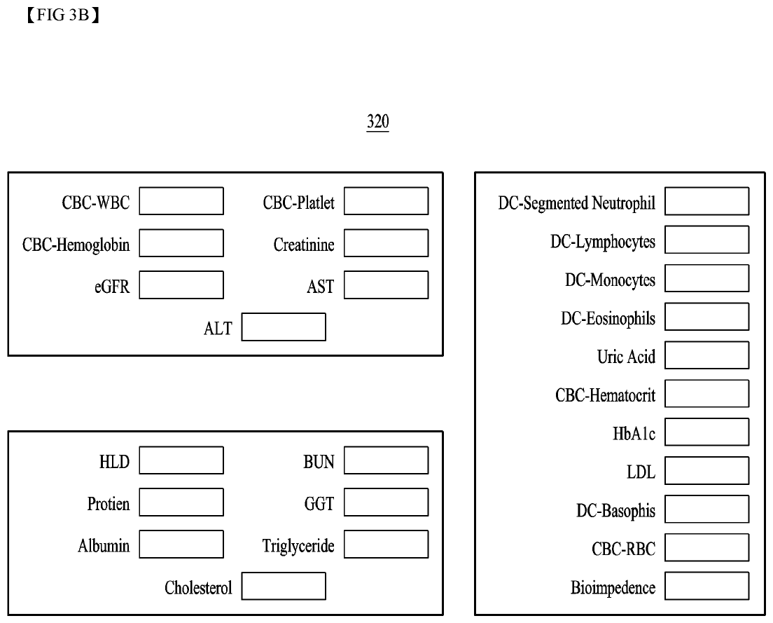

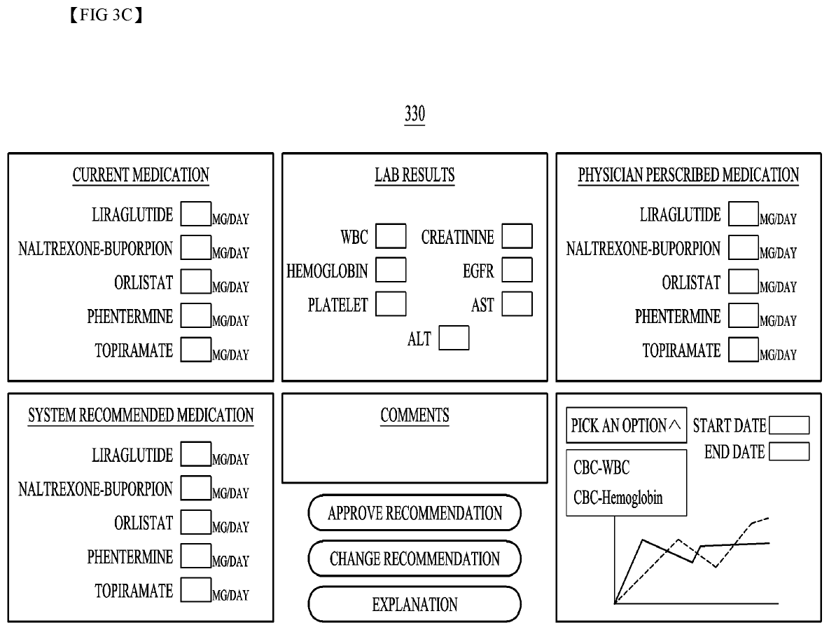

FIGS. 3A to 3C are diagrams for explaining examples of providing information and notification information about a patient in a system for managing pharmacotherapy according to one embodiment.

Referring to FIGS. 3A to 3C, the system for managing pharmacotherapy according to one embodiment may provide information (e.g., profile information and clinical examination information about a patient and pharmacotherapy information) about a patient and notification information for discontinuing or changing a drug prescribed by a medical professional.

A medical professional in charge of a patient may receive information provided from the system for managing pharmacotherapy through a dedicated app or web and use the information in the patient's drug treatment process.

Specifically, as shown in reference numeral 310, the system for managing pharmacotherapy may provide the patient's basic information (name, ID, etc.) and information about the visit date of a medical institution to the terminal of a medical professional.

In addition, as shown in reference numeral 320, the system for managing pharmacotherapy may provide clinical examination information including the measurement value of a vital factor to the terminal of a medical professional.

In addition, as shown in reference numeral 330, the system for managing pharmacotherapy may provide drug prescription information and efficacy analysis results including the measurement value change rate of a vital factor to the terminal of a medical professional.

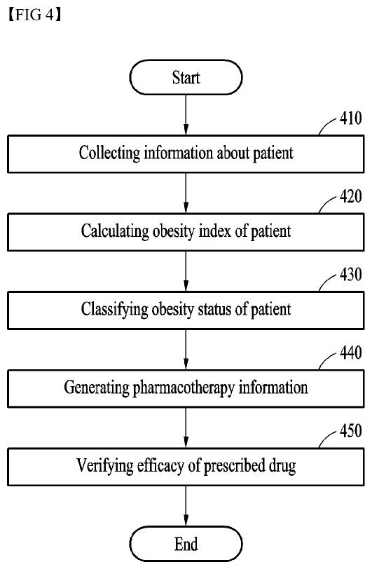

FIG. 4 is a flowchart for explaining a method of operating a system for managing pharmacotherapy according to one embodiment.

FIG. 4 is a flowchart for explaining a method of operating the system for managing pharmacotherapy according to one embodiment described with reference to FIGS. 1 to 3C. Accordingly, in describing FIG. 4, repeated content explained through FIGS. 1 to 3C will be omitted.

Referring to FIG. 4, in step 410, profile information and clinical examination information about a patient may be received by an information receiver.

For example, the profile information may include at least one information of age, gender, height, weight, disease history, disease-related family history, vital signs, physical activity amount, and dietary pattern of a patient.

In addition, the clinical examination information may include results of clinical examination for at least one disease of angina, myocardial infarction, heart failure, thrombosis, coronary artery disease, coronary artery obstructive pulmonary disease, dyspnea, and stroke of a patient.

According to one aspect, the clinical examination information may include evaluation information about at least one of metabolic risk, physical disability, and psychological problems according to the results of clinical examination of the patient.

Next, in step 420, the obesity index of a patient may be calculated based on the profile information by an obesity index calculator.

According to one aspect, in step 420, the obesity index calculator may calculate the body mass index (BMI) of a patient as an obesity index based on the profile information.

Next, in step 430, based on the calculated obesity index and the clinical examination information, the obesity status of a patient may be classified as a preset obesity stage by a status classifier.

According to one aspect, in step 430, when evaluation information is marked as ‘none (Nil)’, the status classifier may classify the obesity status of a patient as stage 0. When evaluation information is marked as ‘mild’, the status classifier may classify the obesity status of a patient as stage 1. When evaluation information is marked as ‘moderate’, the status classifier may classify the obesity status of a patient as stage 2.

In addition, in step 430, when evaluation information is marked as ‘considerable’, the status classifier may classify the obesity status of a patient as stage 3. When evaluation information is marked as ‘serious’, the status classifier may classify the obesity status of a patient as stage 4.

Next, in step 440, by a treatment information generator, pharmacotherapy information corresponding to the classified obesity stage may be generated, and the generated pharmacotherapy information may be provided to the terminal of a medical professional.

According to one aspect, in step 440, pharmacotherapy information including at least one information of long-term medication information and short-term medication information corresponding to the classified obesity stage may be generated by the treatment information generator.

Next, in step 450, by an efficacy verifier, the efficacy of the prescribed drug may be verified based on the measurement values of weight information and vital factors of a patient at a first time point at which a medical professional prescribed a drug to the patient and a second time point at which a preset treatment period has elapsed from the first time point

According to one aspect, in step 450, by the efficacy verifier, based on the verification results of the efficacy of the prescribed drug, notification information for any one of continuation, discontinuation, and change of the prescribed drug may be provided to the terminal of a medical professional.

According to one aspect, in step 450, when a difference between weight information at a second time point and weight information at a first time point exceeds a preset first threshold, the efficacy verifier may generate notification information for continuing a prescribed drug and the dosage of the prescribed drug. When a difference between the weight information does not exceed the first threshold, the efficacy verifier may generate notification information for changing a prescribed drug to another drug and adjusting the dosage of the prescribed drug.

In addition, in step 450, based on a difference between the measurement value of a vital factor at a second time point and the measurement value of a vital factor at a first time point, the efficacy verifier may generate notification information for continuing or discontinuing taking a prescribed drug. Here, the vital factor may include at least one of a first vital factor, a second vital factor, and a third vital factor.

In addition, in the above operation method, steps 420 to 450 may be repeated as many times as the preset number of times per preset treatment cycle by a medical professional.

In conclusion, when the present disclosure is used, by providing pharmacotherapy information based on the classification results of the obesity index and obesity status of a patient, the prescription of drugs that match the patient's obesity status may be supported.

In addition, when the present disclosure is used, the efficacy and effects of a drug prescribed to a patient may be evaluated, and optimizing the drug and dosage prescribed to the patient may be supported.

According to one embodiment, the present disclosure can support the prescription of drugs that match the patient's obesity status by providing pharmacotherapy information based on the classification results of the obesity index and obesity status of a patient.

According to one embodiment, the present disclosure can evaluate the efficacy and effects of a drug prescribed to a patient and support optimizing the drug and dosage prescribed to the patient.

Although the present disclosure has been described with reference to limited embodiments and drawings, it should be understood by those skilled in the art that various changes and modifications may be made therein. For example, the described techniques may be performed in a different order than the described methods, and/or components of the described systems, structures, devices, circuits, etc., may be combined in a manner that is different from the described method, or appropriate results may be achieved even if replaced by other components or equivalents.

Therefore, other embodiments, other examples, and equivalents to the claims are within the scope of the following claims.

### DESCRIPTION OF SYMBOLS

100: SYSTEM FOR MANAGING PHARMACOTHERAPY
110: INFORMATION RECEIVER
120: OBESITY INDEX CALCULATOR
130: STATUS CLASSIFIER
140: TREATMENT INFORMATION GENERATOR
150: EFFICACY VERIFIER

What is claimed is:

1. A system for managing pharmacotherapy, comprising:
an information receiver for receiving profile information and clinical examination information about a patient;
an obesity index calculator for calculating an obesity index of the patient based on the profile information;
a status classifier for classifying obesity status of the patient into a preset obesity stage based on the calculated obesity index and the clinical examination information;
a treatment information generator for generating pharmacotherapy information corresponding to the classified obesity stage and providing the generated pharmacotherapy information to a terminal of a medical professional; and
an efficacy verifier for verifying efficacy of the prescribed drug based on measurement values of weight information and vital factors of the patient at a first time point at which the medical professional prescribed a drug to the patient and a second time point at which a preset treatment period has elapsed from the first time point.

2. The system according to claim 1, wherein the profile information comprises at least one information of age, gender, height, weight, disease history, disease-related family history, vital signs, physical activity amount, and dietary pattern of the patient.

3. The system according to claim 1, wherein the obesity index calculator calculates a body mass index (BMI) of the patient as the obesity index based on the profile information.

4. The system according to claim 1, wherein the clinical examination information comprises results of clinical examination for at least one disease of angina, myocardial infarction, heart failure, thrombosis, coronary artery disease, coronary artery obstructive pulmonary disease, dyspnea, and stroke of the patient.

5. The system according to claim 1, wherein the clinical examination information comprises evaluation information about at least one of metabolic risk, physical disability, and psychological problems according to results of clinical examination of the patient.

6. The system according to claim 5, wherein the status classifier classifies obesity status of the patient as stage 0 when the evaluation information is marked as ‘none (Nil)’, classifies obesity status of the patient as stage 1 when the evaluation information is marked as ‘mild’, and classifies obesity status of the patient as stage 2 when the evaluation information is marked as ‘moderate’.

7. The system according to claim 5, wherein the status classifier classifies obesity status of the patient as stage 3 when the evaluation information is marked as ‘considerable’, and classifies obesity status of the patient as stage 4 when the evaluation information is marked as ‘serious’.

8. The system according to claim 1, wherein the treatment information generator generates the pharmacotherapy information comprising at least one information of long-term medication information and short-term medication information corresponding to the classified obesity stage.

9. The system according to claim 1, wherein the efficacy verifier provides notification information for any one of continuation, discontinuation, and change of the prescribed drug based on verification results of efficacy of the prescribed drug to a terminal of the medical professional.

10. The system according to claim 9, wherein the efficacy verifier generates the notification information for continuing the prescribed drug and dosage of the prescribed drug when a difference between weight information at the second time point and weight information at the first time point exceeds a preset first threshold, and generates the notification information for changing the prescribed drug to another drug and adjusting dosage of the prescribed drug when a difference between the weight information does not exceed the first threshold.

11. The system according to claim 9, wherein the efficacy verifier generates the notification information for continuing or discontinuing taking the prescribed drug based on a difference between a measurement value of a vital factor at the second time point and a measurement value of a vital factor at the first time point,
wherein the vital factor comprises at least one of a first vital factor, a second vital factor, and a third vital factor.

12. The system according to claim 11, wherein the efficacy verifier generates the notification information for continuing the prescribed drug and a dosage of the prescribed drug when a difference between a measurement value of a first vital factor at the second time point and a measurement value of a first vital factor at the first time point does not exceed a preset second threshold.

13. The system according to claim 11, wherein, when a difference between a measurement value of a first vital factor at the second time point and a measurement value of a first vital factor at the first time point exceeds a preset second threshold, and a difference between a measurement value of a second vital factor at the second time point and a measurement value of a second vital factor at the first time point does not exceed the second threshold, the efficacy verifier generates the notification information for continuing or changing a dosage of the prescribed drug.

14. The system according to claim 11, wherein, when a difference between a measurement value of a first vital factor at the second time point and a measurement value of a first vital factor at the first time point exceeds a preset second threshold, a difference between a measurement value of a second vital factor at the second time point and a measurement value of a second vital factor at the first time point exceeds the second threshold, and a difference between a measurement value of a third vital factor at the second time point and a measurement value of a third vital factor at the first time point does not exceed the second threshold, the efficacy verifier generates the notification information for continuing or changing a dosage of the prescribed drug.

15. The system according to claim 11, wherein, when a difference between a measurement value of a first vital factor at the second time point and a measurement value of a first vital factor at the first time point exceeds a preset second threshold, a difference between a measurement value of a second vital factor at the second time point and a measurement value of a second vital factor at the first time point exceeds the second threshold, and a difference between a measurement value of a third vital factor at the second time point and a measurement value of a third vital factor at the first time point exceeds the second threshold, the efficacy verifier generates the notification information for discontinuing taking the prescribed drug.

16. The system according to claim 11, wherein the first vital factor comprises at least one of white blood cells (WBCs), hemoglobins, platelets, creatinine, epidermal growth factor receptor (eGFR), aspartate aminotransferase (AST), and alanine aminotransferase (ALT).

17. The system according to claim 11, wherein the second vital factor comprises at least one of red blood cells (RBCs), 7-glutamyl transferase (GGT), blood urea nitrogen (BUN), cholesterol, high-density lipoprotein (HDL), low-density lipoprotein (LDL), glycated hemoglobin (HbA1c), and bioimpedance.

18. The system according to claim 11, wherein the third vital factor comprises at least one of hematocrit, neutrophils, lymphocytes, monocytes, eosinophils, basophils, uric acid, proteins, albumin, and triglyceride.

19. The system according to claim 1, wherein the treatment information generator generates the pharmacotherapy information comprising initial drug recommendation information for the patient by using a reinforcement learning-based drug recommendation model that uses at least one of the classified obesity stage, the profile information, and the clinical examination information as input.

20. A method of operating a system for managing pharmacotherapy, comprising:
receiving profile information and clinical examination information about a patient by an information receiver;
calculating an obesity index of the patient based on the profile information by an obesity index calculator;
classifying obesity status of the patient into a preset obesity stage based on the calculated obesity index and the clinical examination information by a status classifier;
generating pharmacotherapy information corresponding to the classified obesity stage and providing the generated pharmacotherapy information to a terminal of a medical professional by a treatment information generator; and
verifying, by an efficacy verifier, efficacy of the prescribed drug based on measurement values of weight information and vital factors of the patient at a first time point at which the medical professional prescribed a drug to the patient and a second time point at which a preset treatment period has elapsed from the first time point.
